(19) United States
(12) Patent Application Publication
(10) Pub. No.: US 2008/0292550 A1
(43) Pub. Date: Nov. 27, 2008

(54) Fluorinated 3'-Deoxythymidine Derivatives, a Method for Their Preparation and Their Use

(75) Inventors: Dieter Baerwolff, Panketal (DE); Rolf Lindner, Berlin (DE); Dieter-Ludwig Munz, Berlin (DE); Rainer Michael, Dresden (DE)

Correspondence Address: NORRIS, MCLAUGHLIN & MARCUS, P.A., 875 THIRD AVE, 18TH FLOOR, NEW YORK, NY, 10022, US

(73) Assignees: MAX-DELBRUECK-CENTRUM FUER MOLEKULARE MEDIZIN, Berlin (DE); CHARITE UNIVERSITAETSMEDIZIN BERLIN, Berlin (DE)

(21) Appl. No.: 11/912,425

(22) PCT Filed: Apr. 25, 2006
(86) PCT No.: PCT/EP2006/004228

§ 371 Date: May 6, 2008

(30) Foreign Application Priority Data
Apr. 25, 2005 (DE) 10 2005 019 452.4

(51) Int. Cl. A61K 51/00; C07H 19/067; A61K 31/706

(52) U.S. Cl. 424/1.73; 536/28.55; 514/50

(57) ABSTRACT

The invention relates to new side chain-fluorinated 3′-deoxythymidine derivatives of the general formula I

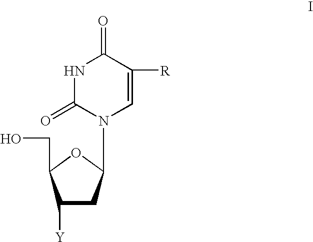

or physiological acceptable esters thereof,
wherein
R═CH₂¹⁸F; CH¹⁸F₂; CH₂F or CHF₂ and
Y═H, N₃ or Hal.

The invention also relates to a simple and efficient method for the preparation of these 3′-deoxythymidines of formula I and to the use of the ¹⁸F-containing compounds of formula I or of a pharmaceutical composition comprising these compounds for the diagnosis of tumors in positron emission tomography (PET).

The invention relates to new side chain-fluorinated 3′-deoxythymidine derivatives of the general formula I

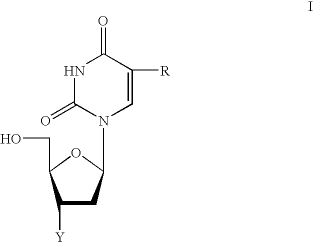

or physiologically acceptable esters thereof,
wherein

### R═CH₂¹⁸F; CH¹⁸F₂; CH₂F or CHF₂ and

### Y═H, N₃ or Hal.

Furthermore, the invention relates to a method for the preparation of these side chain-fluorinated 3′-deoxythymidines of formula I and to the use of the compounds of formula I or of a pharmaceutical composition comprising these compounds for the diagnosis or for the treatment of tumors.

¹⁸F fluorinated compounds of formula I are especially preferred. They are used as diagnostic tools in positron emission tomography (PET) at tracer doses by administering them or compositions containing them in effective amounts to identify susceptible tumors in biopsy specimens or via external imaging.

For diagnosis in oncology, as well as in neurology, psychiatry, cardiology and orthopedics, the positron emission tomography (PET) has gained more and more acceptance. It reflects pathological conditions in spatially confined tissue concentrations of radioactive tracers, which deviate from normal physiology.

PET is based on the β⁺-decay of primarily light-weight isotopes (¹⁵O, ¹³N, ¹¹C, ¹⁸F), wherein a proton undergoes decay to form a neutron, liberating a positron and a neutrino. Being an antiparticle, the positron reacts almost immediately with an electron from the surrounding matter, with annihilation of both particles and emission of two γ-quanta moving apart at 180° with an energy of 511 keV each, which are measured in a detector ring (PET camera) and allow imaging of the distribution of a PET pharmaceutical agent (tracer) in an organism. Owing to its relatively long half-life (109.7 min), ¹⁸F is the isotope most frequently used, and the tracer most frequently used—representing a model substance at the same time—is ¹⁸F-fluorodeoxyglucose (¹⁸F-FDG). ¹⁸F-FDG shows increased glucose conversion, which is present in most tumors and centers of inflammation, in the form of more intensely colored areas in a PET camera image.

In oncology, one problem is lacking discrimination of the ¹⁸F-FDG tracer between tumors and centers of inflammation. Some tumors show no increase of glucose conversion. Therefore, more recent tracer developments focus on radioactive labelling of deoxynucleosides whose metabolism, being part of DNA synthesis, allows a more direct representation of increased proliferation (cell division rate) that is present in tumors. One milestone in these developments has been ¹⁸F labelling of thymidine in the 3′ position (¹⁸F-FLT) and the use thereof in PET by Shields et al. in 1998 (Shields AF: PET imaging with ¹⁸F-FLT and thymidine analogs: promise and pitfalls. J Nucl Med 2003; 44(9): 1432-4). For routine use of ¹⁸F-FLT, however, the synthesis thereof is too complex.

Therefore, it is the aim of the present invention to provide new ¹⁸F tracers being suitable in PET. A further object of the invention is to provide a simple and efficient method for the preparation of these compounds, whereby the fluorination must meet the demand of allowing the preparation within the half-life time of ¹⁸fluorine.

Surprisingly, it was found that ¹⁸F side chain-fluorinated monofluoro and difluoro nucleoside compounds are stored in tumors, thus being highly suitable as PET tracers. At present a simple method for introducing one or two fluorine atoms in the side chain of the thymine moiety (in the 5-methyl position) is not known. The present invention solves this problem and a new method of preparation of such desired side chain-fluorinated compounds uses an easily accessible precursor compound for fluorination.

Therefore, subject matter of the present invention are new 3′-deoxythymidine derivatives which are monofluoro or difluoro side chain-fluorinated compounds, fluorinated in the 5-methylposition of the pyrimidine (thymine) moiety and having the formula I

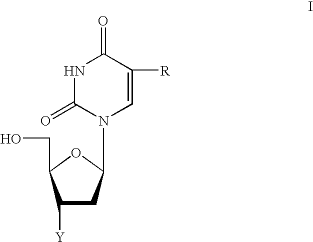

or physiologically acceptable esters thereof,
wherein

### R═CH₂¹⁸F or CH¹⁸F₂; CH₂F or CHF₂ and

### Y═H, N₃ or Hal.

According to the invention a physiologically acceptable ester is an ester which does not inhibit the receptivity of the compounds by the cells, such as for instance the acetyl or the formyl ester. According to the invention Hal in formula I means F, Cl or Br, preferably F.

As already described it was found that ¹⁸F side chain-fluorinated monofluoro and difluoro nucleoside compounds are stored in tumors, thus being highly suitable as PET tracers. Therefore, especially ¹⁸F fluorinated compounds of formula I are subject matter of the present invention, wherein R═CH₂¹⁸F or CH¹⁸F₂. Furthermore subject matter of the invention are the preferred ¹⁸F-fluorinated 3′-deoxythymidine derivatives having a Y residue being H, N₃ or Hal, whereby the 5,5-difluoro compounds having R═CH¹⁸F₂ are the most preferred compounds.

Compounds of formula I having ¹⁸F in the residue R and Y═H, N₃ or F are particularly suitable in PET, namely 5-¹⁸F-fluorinated 3′-deoxythymidines, 5-¹⁸F-fluorinated 3′-deoxy-3′-azidothymidines and 5-¹⁸F-fluorinated 3′-deoxy-3′-fluorothymidines. Especially preferred are the 5,5-¹⁸F difluoro compounds of formula I, namely ¹⁸F-5,5-difluoro-3′-deoxythymidine (α,α-¹⁸F₂-dT), ¹⁸F-5,5-difluoro-3′-deoxy-3′-azidothymidine (α,α-¹⁸F₂-AZT) and ¹⁸F-5,5-difluoro-3′-deoxy-3′-fluorothymidine (α,α-¹⁸F₂-FLT). The most preferred compound is the 5,5-¹⁸F-difluoro-3′-deoxy-3′-azidothymidine (α,α-8F₂-AZT) which is the compound of formula I with Y═N₃ and R═CH¹⁸F₂.

According to the present invention the new method for preparation of the compounds of the invention uses an easily accessible precursor compound for fluorination. Surprisingly, a 5-monobromo and 5,5-dibromo-3′-deoxythymidine derivative of formula II is a good starting precursor material for a following fluorination.

This reaction proceeds according to the following scheme:

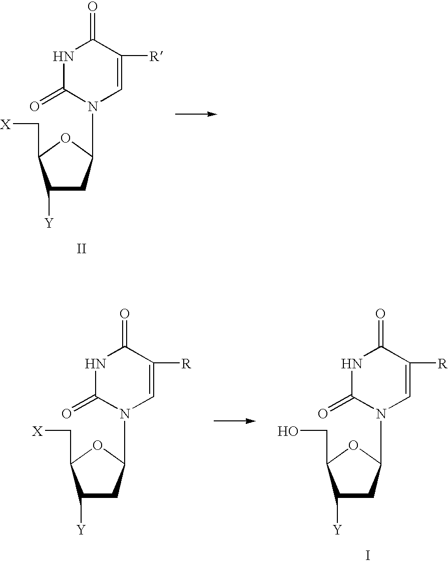

wherein

### R′═CH₂Br or CHBr₂

X=a protective group, preferably an O-acyl group, for example O-acetyl, O-benzoyl, O-p-nitrobenzoyl, O-palmitoyl or O-trifluoroacetyl group; and
R and Y have the same meaning as in formula I.

One essential advantage and, at the same time, a precondition for the usability of these compounds is that the preparation of compounds of formula II is possible by simple synthesis from readily accessible starting materials. The compounds of formula II can be produced from known 3′-deoxythymidine derivatives by free-radical bromination, preferably in esters such as dialkyl carbonates, in a relatively simple synthesis. These starting compounds, for example thymidine (dT), azidothymidine (AZT) or fluorothymidine (FLT) are commercial available and their pharmacological/toxicological properties are well-known.

For the preparation, the hydroxyl groups of these starting compounds are protected preferably with an acyl group at the 5′ position, for example with an acetyl, a benzoyl, a p-nitrobenzoyl, a palmitoyl or a trifluoroacetyl group. Then, the protected compounds are reacted with bromine or with N-bromine succinimide to the resulting 5-monobromo or 5,5-dibromo derivatives of formula II. The proportion of the bromination agent defines the degree of halogenation.

According to the invention a preferred protective group in 5′ position is the trifluoroacetyl group which otherwise is scarcely used with nucleosides due to its instability, but surprisingly is best suited in the method according to the invention.

The following fluorination is effected in such a way that an excess 5-bromo-3′deoxythymidine derivative (5-monobromo or 5,5-dibromo-3′-deoxythymidine derivative) which is protected in the 5′ position is reacted with a fluorinating agent in an inert solvent, for example acetonitrile and, (if necessary) following removal of the protective group, the reaction product is taken up in a solvent and eluted over silica gel using an inert solvent, for example ethyl acetate, acetone or ether or a mixture thereof.

In an embodiment of the invention the ¹⁸fluorination is carried out with a dried ¹⁸F salt as fluorinating agent. The preferred ¹⁸F salt is K¹⁸F and the process is reacted preferably in the presence of a fluorination catalyst, preferably Kryptofix. The bromine-fluorine exchange of the invention using ¹⁸-fluoride results in labelling of the employed thymidine derivatives. One essential issue in achieving high yields is complete drying of the fluorinating agent.

To remove unreacted starting material and byproducts, the removal of the protective group is followed by quenching (if necessary), preferably with ethanol and/or triethylamine. As the case may be thereafter, all volatile products are evaporated preferably at 90° C.

Half the battle of the inventive method list that thereby the objective of completing synthesis, purification and conditioning of the final product is achieved within just under 2 hours, which approximately corresponds to the half-life of ¹⁸-fluorine (109 minutes).

In a preferred achievement of the invention an especially applicable tracer has been found in 5, 5-18F-difluoro-3′-deoxy-3′-azidothymidine (α,α-¹⁸F₂-AZT) which can be prepared as shown above in a relatively simple synthesis by nucleophilic halogen exchange in the thymine side chain (5-position).

For preparation of non-labelled compounds of formula I tetrabutylammonium fluoride is used as fluorinating agent. The non-labelled compounds of formula I with R═CH₂F or CHF₂ are also an object of the present invention.

In a further embodiment of the invention, the compounds of the invention are used for the diagnosis and treatment of humans suffering from tumors, in particular, from cancerous tumors. However, it will be appreciated that the invention can be used in other mammals, for example in veterinary applications, and in non-mammalian species having a similar biochemical/physiological basis for pathological conditions.

A pharmaceutical composition which comprises at least one compound of formula I and a pharmaceutically acceptable carrier substance is a further object of the present invention. Because it is preferred to administer the compounds of the invention by injection or infusion, the carrier substance is preferably a buffer solution, such as for instance PBS.

The ¹⁸F compounds of the invention are suitable as diagnostics in a method of performing positron emission tomography (PET), especially for tumors. The PET with the compounds of the invention is especially useful in the detection of cancer, because metastatic tumors that may not be visualized by other imaging techniques are detectable.

The new compounds of the invention are especially useful as PET tracers to detect recurrent brain tumors and cancers of the lung, colon, breast, lymph nodes, skin, and other organs, for instance blood, small intestine, spleen, liver, heart, kidney and bone. It could be demonstrated that the new compounds can be stored especially in lung tumors and in melanoma. Furthermore, it was found that the new compounds (owing to their higher lipophilicity) are capable of passing the blood-brain barrier. An imaging of brain tumors is also possible by means of the new ¹⁸F-tracers.

Therefore, a further embodiment of the invention is a method of performing positron emission tomography (PET) in a mammal by administering the nucleosides of formula I containing ¹⁸F or the pharmaceutical composition comprising these compounds. In a preferred embodiment 5,5-¹⁸F-difluoro-3′-deoxy-3′-azidothymidine is used. The method is preferably carried out to detect tumors. The new radiopharmaceutical of formula I are preferably given by intravenous injection or by infusion a few minutes before the PET procedure. During the scan, the patient lies comfortably; the only discomfort involved may be the pinprick of a needle used to inject the radiopharmaceutical.

The PET scan of a healthy organ or body part will yield images without contrasting regions, because the radiolabeled compound will have been metabolised at the same rate. The PET scan of a diseased organ or body part however, will yield images showing contrasting regions, because the radiolabeled compound will not have been metabolized at the same rate by the healthy and diseased cells.

The non-labelled compounds of formula I can be used for the preparation of a composition for therapeutic purposes, e.g. as a cytostatic agent and in the treatment of dermal diseases. The non-labelled compounds and therapeutic compositions comprising these compounds are also object of the present invention. These compounds and compositions comprising these compounds are easily administered by different modes known in the art and can be given in dosages that are safe and provide tumor inhibition at the relevant sites.

Advantages of the invention are as follows:
Being a readily available starting material with well-known toxicological properties, namely 3′-deoxythymidine derivatives were found suitable for precursor synthesis.
The starting precursor compounds, the 5-bromo-3′-deoxythymidines, can be produced from accordant 3′-deoxythymidine derivatives by free-radical bromination in a relatively simple synthesis.
Especially the ¹⁸F-difluoro labelling of a 5,5-dibromo-3′-deoxythymidine derivative can be accomplished under optimized synthesis parameters with conversions of up to 60%.
It is possible to synthesize the desired compound in radiochemical yields of up to 5%, which product is sufficiently stable for further investigations and free of fluoride.

The invention will be illustrated in more detail by using 5-bromo azidothymidine derivatives as precursor compounds to get compounds of formula I having Y═N₃, and R═CH₂¹⁸F or CH¹⁸F₂.

### 1. Fluorination of 3′-deoxy-3′-azidothymidine derivatives

### 1.1. Precursor: 5-Monobromo-3′-deoxy-3′-azidothymidine (α-Br-AZT)

5-bromo-3′-deoxy-3′-azidothymidine was initially selected produced from azidothymidine (AZT) as a starting material which is commercial available, and its pharmacological/toxicological properties are well-known. To facilitate saponification, the acetate protective group was replaced by trifluoroacetate. Saponification was easily achieved using MeOH, with no additional heating. The following fluorination results in α-¹⁸F-AZT.

### 1.2. Removal of Fluoride

To achieve removal of unreacted fluoride the final product (α-¹⁸F-AZT) was transferred into an isotonic NaCl solution and the fluoride content was so reduced below 3%. To this end, a number of HPLC experiments, as well as experiments using various eluents and various purification cartridges were carried out.

### 1.3. Precursor 5,5-Dibromo-3′-deoxy-3′-azidothymidine (α,α-Br₂-AZT)

By means of the above precursor (α,α-Br₂-AZT) it was possible to achieve substantially higher conversion (up to 60%) right from the start.

¹⁸F ions still present were successfully removed with silica gel in mixtures of organic solvents (below 0.3% fluoride). The best results were achieved with a mixture of 50% acetone and 50% ether. The yields, being 3 to 5%, were sufficient for further investigations.

Following evaporation of the methanol (after saponification), the final product was dissolved in an acetone/ether mixture and eluted with ethyl acetate over silica gel. The ethyl acetate is removed by evaporation under a stream of helium at room temperature. The residue is taken up in isotonic NaCl solution and is ready for further investigations (enzymatic tests, animal experiments). The resulting α,α-¹⁸F₂-AZT is stable in isotonic NaCl solution at room temperature (e.g., increase of the F ion concentration from 3 to 4.5% within 1 hour).

### 2. Improving the Stability by Quenching

To improve the stability of the final product, a quenching step using triethylamine (900 μl of TEA) was introduced as last step of the synthesis. As a result of quenching, unreacted precursor and unstable ¹⁸F-labelled substances are converted into polar compounds which are insoluble or sparingly soluble in organic solvents, being removed in appropriate cartridges. In this way, transfer thereof into the final vessel is largely excluded.

Using serum in an incubator at 37° C. it could demonstrate markedly improved stability of α,α-¹⁸F₂-AZT. A similar result was achieved with serum at room temperature (21° C.). In isotonic NaCl solution at 21° C., α,α-¹⁸F₂-AZT showed scarcely any increase of the fluoride concentration over a period of 4 hours. In addition, a significant reduction of the initial fluoride content could be achieved.

### EXAMPLES

### Example 1

### Preparation of 5,5-Dibromo-3′-deoxy-3-azidothymidine (α,α-Br₂-AZT)

3.63 g (10 mmol) 3′-deoxy-3′-azido-5′-trifluoroacetyl-thymidine are solved in 100 ml dimethylcarbonate and heated by a 250 W photo-lamp under reflux in an argon-atmosphere. During this heating 1.1 ml (22 mmol) bromine are blowed into the solution (1-2 hours). The progress of the reaction is controlled by TLC (Merck, silicagel-F 254, ethylacetate). At the end of the reaction no starting material is detectable. The left solution is evaporated in vacuum to dryness and coevaporated with dimethylcarbonate several times. The α,α-Br₂-AZT is stored with argon at 0° C. in the dark for weeks. During the procedure moisture should be avoided in all steps.

### Example 2

### Preparation of 5,5-¹⁸F-difluoro-3′-deoxy-3′-azidothymidine (α,α-F₂-AZT)

¹⁸F is obtained from ¹⁸O-water by proton irradiation in an RDS 111 cyclotron of Charité, Berlin, and transferred into a computer-controlled synthesis module. Separation of ¹⁸F from ¹⁸O-water is effected in an ion exchange column by elution with 0.5 ml of 0.2% K₂CO₃ into the reaction vessel. Thereafter, this is dried in two steps using 1 ml and 2 ml, respectively, of acetonitrile at 105° C. 20 mg of fluorination catalyst, Kryptofix K2.2.2., in 1 ml acetonitrile is added in the second drying step. Drying is followed by addition of 20-100 mg of α,α-Br₂-AZT in acetonitrile. The reaction is carried out under 0.25 MPa He for 10 min. After the reaction, the trifluoroacetyl protective group is saponified with methanol, and undesirable monofluoroazidothymidine and unreacted α,α-Br₂-AZT are quenched with triethylamine. To this end, 100 μl of methanol and 900 μl of triethylamine are added together. The methyl trifluoroacetate being formed is evaporated at 90° C., together with acetonitrile still present in the reactor and unreacted triethylamine and methanol. Subsequently, the reaction product is taken up in 0.5 ml of a mixture of 50% acetone and 50% diethyl ether and eluted with ethyl acetate over a silica gel cartridge (about 200 mg of silica gel). Finally, the ethyl acetate is removed from the eluate in a stream of helium at room temperature, and the residue, consisting of 5,5-¹⁸F-difluoro-3′-deoxy-3′-azidothymidine, is taken up in isotonic NaCl solution.

### Example 3

### Animal Experiments

In the animal experiments, tumor-bearing nude mice (non-parvicellular lung tumor) each received about 0.5 MBq of non-quenched ¹⁸F-difluoroazidothymidine i.v., and the organ activities were measured after 30 min and 60 min, respectively. As a result, activity was stored in the tumor, in the spleen and in the colon, whereas the activity markedly decreased in all the other organs (blood, heart, lungs, liver, stomach, small intestine, kidneys, and muscle sample), particularly in muscle tissue. The tumor/tissue radioactivity quotients reflect the storage of ¹⁸F-5,5-difluoro-3′-deoxy-3′-azidothymidine in the tumor in relation to the most important organs. Absorption of ¹⁸F was particularly high in bone tissue, but showed a clear tendency to decline after 60 minutes. Presumably, the high absorption in bone was caused by F ions. Storage in the colon occurs as a result of tracer elimination via the feces. Marked storage of ¹⁸F-FLT in the spleen of mice has also been observed by Wagner et al. (Cancer Research 2003; 63: 2681-2687), and is caused by the high nucleotide conversion in lymphocyte production.

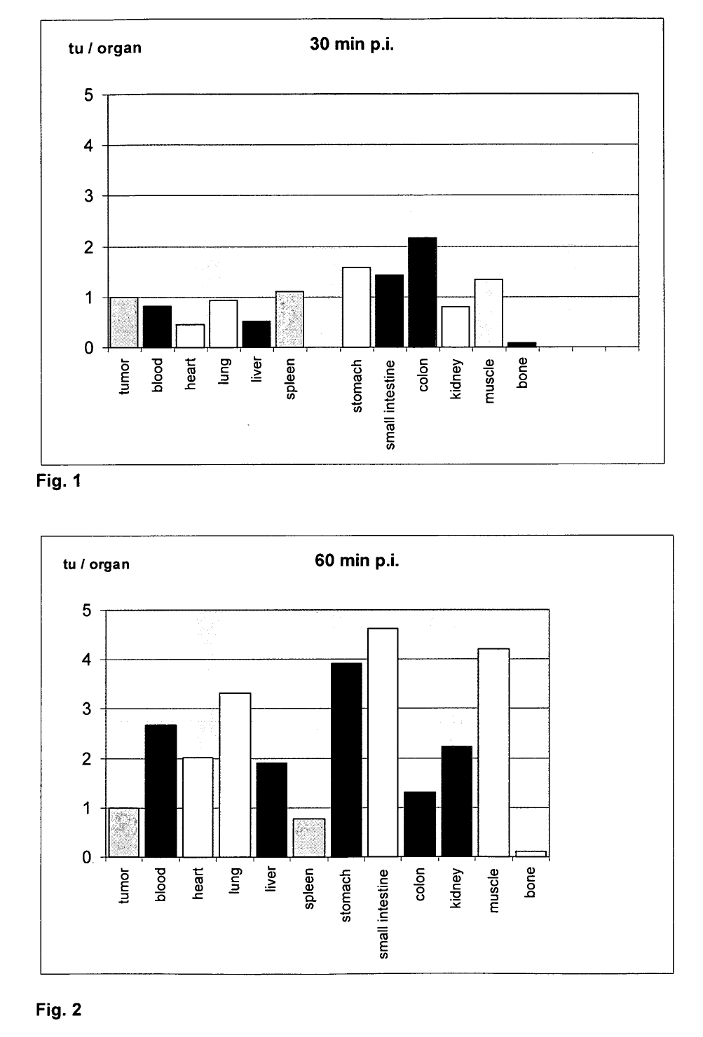

In further animal experiments, melanoma-bearing nude mice each received about 0.5 MBq of quenched α,α-¹⁸F₂-AZT i.v., and the organ activities were measured after 30 (FIG. 1), 60 (FIG. 2) and 90 min. Using the improved synthetic product, it was possible to achieve high tumor/organ radioactivity ratios after 60 min. Storage of ¹⁸F in bone was lower. A prognosis as to the tumor/background ratio in a tumor image can be made by means of the tumor/blood radioactivity ratio of a tracer in a tumor model. A ratio of 3.2 was reached in our tumor model after 60 min. Using 0.1 MBq of ¹⁸F-FLT, Wagner et al., 2003, have obtained a tumor/blood radioactivity ratio of about 7 in a B cell lymphoma mouse model, and Barthel et al (Cancer Res. 2003; 63(13): 3791-8) have obtained 1.8 with about 2.5 MBq in a fibrosarcoma mouse model. Surprisingly, it appeared that α,α-¹⁸F₂-AZT, unlike ¹⁸F-FLT, passes the blood-brain barrier—probably due to higher lipophilicity.

In summary, in investigations conducted so far, it has been demonstrated that the tracer 5,5-¹⁸F-difluoro-3′-deoxy-3′-azidothymidine (α,α-¹⁸F₂-AZT) which can be produced in a relatively simple synthesis by nucleophilic halogen exchange in the thymine side chain, and is stored in lung tumor and in melanoma in a mouse model, allows PET imaging of melanoma in a mouse model. The 5,5-dibromo-3′-deoxy-3′-azidothymidine precursor was obtained by means of a synthesis which is also relatively simple. This result represents a substantial expansion of the diagnostic options for tumors. In comparison with the ¹⁸F-labelled thymidine analog 3′-¹⁸F-fluoro-3′-deoxythymidine (¹⁸F-FLT), which is the only one used so far, another advantage is that α,α-¹⁸F₂-AZT, owing to its higher lipophilicity, is capable of passing the blood-brain barrier, so that imaging of brain tumors is possible by means of the above tracer, too.

FIG. 1 exhibits organ activities after 30 min using 0.5 MBq of quenched α,α-¹⁸F₂-AZT

FIG. 2 exhibits organ activities after 60 min using 0.5 MBq of quenched α,α-¹⁸F₂-AZT

1-24. (canceled)

25. A fluorinated 3′-deoxythymidine derivative of general formula I 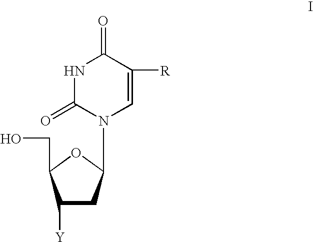
or a physiologically acceptable ester thereof,
wherein
R═CH₂¹⁸F or CH¹⁸F₂ or CHF₂ and
Y═H, N₃ or Hal

26. The ¹⁸F-fluorinated 3′-deoxythymidine derivative according to claim 25, wherein R═CH₂¹⁸F or CH¹⁸F₂.

27. The ¹⁸F-fluorinated 3′-deoxythymidine derivative according to claim 26 wherein Y═H, N₃ or F.

28. The ¹⁸F-fluorinated 3′-deoxythymidine derivative according to claim 25 wherein R═CH¹⁸F₂.

29. The ¹⁸F-fluorinated 3′-deoxythymidine derivative according to claim 25 wherein Y═N₃ and R═CH¹⁸F₂.

30. The fluorinated 3′-deoxythymidine derivative according to claim 25, wherein R═CHF₂.

31. A method for the preparation of a 3′-deoxythymidine derivative of general formula I according to claim 25, characterized in that a fluorinating agent is reacted in an inert solvent with an excess 5-bromo-3′-deoxythymidine derivative of formula II which is protected in 5′ position, and, following, if necessary, the removal of the protective group according to the following scheme 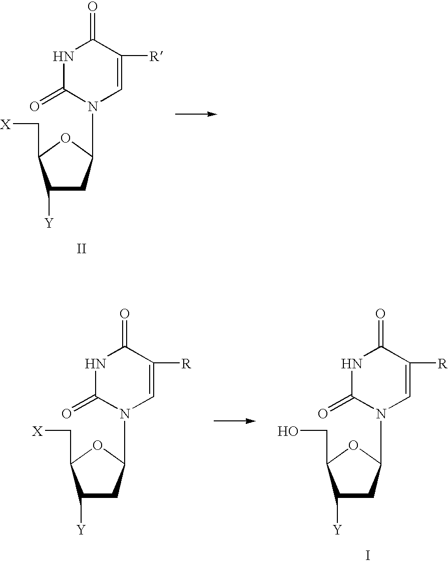
wherein R′═CHBr₂ or CH₂Br, X is a protective group, preferably an O-acyl group, and R and Y have the same meaning as in formula I.

32. The method according to claim 31, characterized in that an ¹⁸F-fluorinating agent is reacted with excess 5-monobromo-3′-deoxythymidine derivative.

33. The method according to claim 31, characterized in that an ¹⁸F-fluorinating agent is reacted with excess 5,5-dibromo-3′-deoxythymidine derivative.

34. The method according to claim 31, characterized in that a dried ¹⁸F salt is used as fluorinating agent, preferably K¹⁸F.

35. The method according to claim 31, characterized in that the fluorination is carried out in the presence of a fluorination catalyst, preferably in the presence of Kryptofix.

36. The method according to claim 31, characterized in that tetrabutylammonium fluoride is used as fluorinating agent for the preparation of non-labelled compounds of formula I.

37. The method according to claim 31, characterized in that the protective group in 5′ position is the trifluoroacetyl group.

38. The method according to claim 31, characterized in that the protective group is removed, preferably with methanol.

39. The method according to claim 31, characterized in that compound I is eluted over silica gel using an inert solvent, preferably ethyl acetate.

40. The method according to claim 31, characterized in that the compound I is quenched whereby the reaction product is taken up in a solvent preferably ethanol and/or triethylamine, and preferably subsequent evaporation of all volatile reaction components.

41. A pharmaceutical composition comprising at least one compound of claim 25 and a pharmaceutically acceptable carrier material.

42. A method of performing positron emission tomography (PET) in a mammal by administering the ¹⁸F containing 3′-deoxythymidine derivatives according to claim 25.

43. The method according to claim 42, characterized in that 5,5-¹⁸F-difluoro-3′-deoxy-3′-azidothymidine is used.

44. The method according to claim 42 for the detection of tumors, preferably for detection of lung tumors, brain tumors or melanoma.
